(19) United States
(12) Patent Application Publication
(10) Pub. No.: US 2018/0279935 A1
(43) Pub. Date: Oct. 4, 2018

(54) METHOD AND SYSTEM FOR DETECTING FREQUENCY DOMAIN CARDIAC INFORMATION BY USING PUPILLARY RESPONSE

(71) Applicants: Sangmyung University Industry-Academy Cooperation Foundation, Seoul (KR); Center Of Human-Centered Interaction For Coexistence, Seoul (KR)

(72) Inventors: Min Cheol Whang, Goyang-si (KR); Sang In Park, Goyang-si (KR); Myoung Ju Won, Cheonan-si (KR); Dong Won Lee, Seongnam-si (KR)

(73) Assignees: Sangmyung University Industry-Academy Cooperation Foundation, Seoul (KR); Center Of Human-Centered Interaction For Coexistence, Seoul (KR)

(21) Appl. No.: 15/887,364

(22) Filed: Feb. 2, 2018

(30) Foreign Application Priority Data
Feb. 17, 2017 (KR) 10-2017-0021522
Nov. 7, 2017 (KR) 10-2017-0147610

(51) Int. Cl. A61B 5/16; A61B 5/024; A61B 5/0205; A61B 5/0476

(52) U.S. Cl. CPC A61B 5/163; A61B 5/02405; A61B 3/112; A61B 5/0476; A61B 5/0205

(57) ABSTRACT

Provided are a method and system for detecting frequency-domain cardiac information, the method comprising: acquiring moving images of a pupil from a subject; extracting a pupil size variation (PSV) from the moving images; extracting a heart rate variability (HRV) spectrum by performing a processing procedure including frequency-analysis of HRV; and calculating power of at least one of a plurality of frequency bands by analysis of the HRV spectrum.

### CROSS-REFERENCE TO RELATED APPLICATION

This application claims the benefit of Korean Patent Application Nos. 10-2017-0021522, filed on Feb. 17, 2017, and 10-2017-0147610, filed on Nov. 7, 2017, in the Korean Intellectual Property Office, the disclosures of which are incorporated herein in their entirety by reference.

### BACKGROUND

### 1. Field

One or more embodiments relate to a method of detecting physiological information by using a pupillary response, and a system using the method, and more particularly, to a method of detecting frequency-domain cardiac information from a pupil size variation, and a system using the method.

### 2. Description of the Related Art

In vital signal monitoring (VSM), physiological information can be acquired by a sensor attached to a human body. Such physiological information includes electrocardiogram (ECG), photo-plethysmograph (PPG), blood pressure (BP), galvanic skin response (GSR), skin temperature (SKT), respiration (RSP) and electroencephalogram (EEG).

The heart and brain are two main organs of the human body and analysis thereof provide the ability to evaluate human behavior and obtain information that may be used in response to events and in medical diagnosis. VSM may be applicable in various fields such as ubiquitous healthcare (U-healthcare), emotional information and communication technology (e-ICT), human factor and ergonomics (HF&E), human computer interfaces (HCIs), and security systems.

Regarding ECG and EEG, sensors attached to the body are used to measure physiological signals and thus, may cause inconvenience to patients. That is, the human body experiences considerable stress and inconvenience when using sensors to measure such signals. In addition, there are burdens and restrictions with respect to the cost of using the attached sensors and to the movement of the subject, due to attached sensor hardware.

Therefore, VSM technology is required in the measurement of physiological signals by using non-contact, non-invasive, and non-obtrusive methods while providing unfettered movement at low cost.

Recently, VSM technology has been incorporated into wireless wearable devices allowing for the development of portable measuring equipment. These portable devices can measure heart rate (HR) and RSP by using VSM embedded into accessories such as watches, bracelets, or glasses.

Wearable device technology is predicted to develop from portable devices to “attachable” devices shortly. It is also predicted that attachable devices will transition to “edible” devices.

VSM technology has been developed to measure physiological signals by using non-contact, non-invasive, and non-obtrusive methods that provide unfettered movement at low cost. While VSM will continue to advance technologically, innovative vision-based VSM technology is required to be developed also.

### SUMMARY

One or more embodiments include a system and method for inferring and detecting human vital signs by using a non-invasive and non-obstructive method at low cost.

In detail, one or more embodiments include a system and method for detecting frequency-domain cardiac information by using a pupillary response or pupil size variation.

Additional aspects will be set forth in part in the description which follows and, in part, will be apparent from the description, or may be learned by practice of the presented embodiments.

According to one or more exemplary embodiments, the method of detecting frequency-domain cardiac information comprises acquiring moving images of a pupil from a subject; extracting a pupil size variation (PSV) from the moving images; extracting a heart rate variability (HRV) spectrum by performing a processing procedure including a frequency-analysis of the PSV; and calculating at least one power of at least one of a plurality frequency bands from the HRV spectrum.

According to one or more exemplary embodiments, a system performing the method comprises a video capturing unit configured to capture the moving images of the subject; and a computer architecture based analyzing system, including analysis tools, configured to process, analyze the moving images, and calculate at least one of the powers.

According to one or more exemplary embodiments, the processing procedure includes filtering using a band pass filter (BPF) and frequency analysis using fast Fourier transformation (FFT).

According to one or more exemplary embodiments, the plurality of frequency bands are the same as a plurality of bands extracted from electrocardiogram (ECG) signals.

According to one or more exemplary embodiments, the plurality of frequency bands include at least one of a very low frequency (VLF) in a range of 0.0033 Hz to 0.04 Hz, a low frequency (LF) in a range of 0.04 Hz to 0.15 Hz, and a high frequency (HF) in a range of 0.15 Hz to 0.4 Hz.

### BRIEF DESCRIPTION OF THE DRAWINGS

These and/or other aspects will become apparent and more readily appreciated from the following description of the embodiments, taken in conjunction with the accompanying drawings in which:

FIG. 1 shows a procedure for selecting a representative of sound stimuli used in an example test, according to one or more embodiments;

FIG. 2 shows an experimental procedure for measuring the amount of movement in an upper body, according to one or more embodiments;

FIG. 3 is a block diagram for explaining an experimental procedure, according to one or more embodiments;

FIG. 4 shows a procedure for detecting a pupil region, according to one or more embodiments;

FIG. 5 schematically shows processes of obtaining frequency-domain cardiac parameters from pupil diameter signals of a pupil size variation (PSV)) and electrocardiogram (ECG) signals;

FIG. 6 shows a sample of averages of amounts of movement in an upper body;

FIGS. 7A and 7B show examples of processing for extracting a heart rate variability (HRV) from the PSV and ECG signals;

FIG. 8 shows a comparison example of cardiac frequency indexes extracted from the pupillary response and ECG signals in a motionlessness condition (MNC);

FIG. 9 shows a comparison example of the cardiac frequency index extracted from the pupillary response and ECG signals in a natural movement condition (NMC);

FIG. 10 shows an infrared webcam system for capturing pupil images, according to one or more embodiments.

FIG. 11 shows an interface screen of a real-time system, according to one or more embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Hereinafter, a method and system for inferring and detecting physiological signals according to the present inventive concept is described with reference to the accompanying drawings.

The invention may, however, be embodied in many different forms and should not be construed as being limited to the embodiments set forth herein; rather, these embodiments are provided so that this disclosure will be thorough and complete, and will fully convey the concept of the invention to those of ordinary skill in the art. Like reference numerals in the drawings denote like elements. In the drawings, elements and regions are schematically illustrated. Accordingly, the concept of the invention is not limited by the relative sizes or distances shown in the attached drawings.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, numbers, steps, operations, elements, and/or components, but do not preclude the presence or addition of one or more other features, numbers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present application, and will not be interpreted in an overly formal sense unless expressly so defined herein.

The embodiments described below involve processing frequency-domain cardiac parameters from a pupillary response which is obtained from video information.

The present invention, which may be sufficiently understood through the embodiments described below, involves extraction of frequency-domain cardiac parameters from the pupillary response or pupil size variation by using a vision system equipped with a video camera such as a webcam without any physical restriction or psychological pressure on the subject. In particular, the pupillary response is detected from the image information and frequency-domain cardiac parameters are extracted from the detected pupillary response.

In an experiment of the present invention, the reliability of the frequency-domain cardiac parameters extracted from the pupil size variation (PSV) acquired through moving images was compared with the ground truth signal by ECG sensors.

Experiments in relation to the present invention were performed by using video equipment, and a computer architecture based analyzing system for processing and analyzing the moving images which included analysis tools provided by software. The system according to exemplary embodiments was developed using Visual C++ 2010 and OpenCV 2.4.3. The signal processing function for fast Fourier transformation (FFT), band-pass filter (BPF), etc. was provided by Lab VIEW 2010.

### Experimental Stimuli

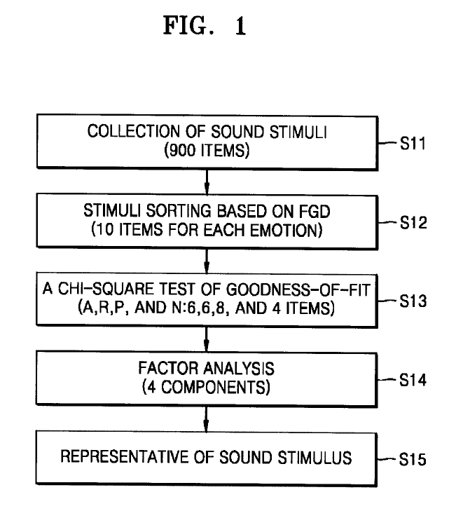

In order to cause variation in a physiological state, this experiment used sound stimuli based on the Russell's cir-complex model (Russell, 1980). The sound stimuli included a plurality of factors, including arousal, relaxation, positive, negative, and neutral sounds. The neutral sound was defined by an absence of acoustic stimulus. The steps for selecting sound stimuli are shown in FIG. 1 and listed as follows:

(S11) Nine hundred sound sources were collected from the broadcast media such as advertisements, dramas, and movies.

(S12) The sound sources were then categorized into four groups (i.e., arousal, relaxation, positive, and negative). Each group was comprised of 10 commonly selected items based on a focus group discussion for a total of forty sound stimuli.

(S13) These stimuli were used to conduct surveys for suitability for each emotion (i.e., A: arousal, R: relaxation, P: positive, and N: negative) based on data gathered from 150 subjects that were evenly split into 75 males and 75 females. The mean age was 27.36 years±1.66 years. A subjective evaluation was required to select each item for the four factors, which could result in duplicates of one or more of the items.

(S14) A chi-square test for goodness-of-fit was performed to determine whether each emotion sound was equally preferred. Preference for each emotion sound was equally distributed in the population (arousal: 6 items, relaxation: 6 items, positive: 8 items, and negative: 4 items) as shown in Table 1.

Table 1 shows the chi-square test results for goodness-of-fit in which the items selected for each emotion are based on comparisons of observation and expectation values.

TABLE 1

| | | |
|---|---|---|
| N | Chi-Square | Sig. |

Arousal

| | | | |
|---|---|---|---|
| arousal 1 | 150 | 83.867 | .000 |
| arousal 2 | 150 | 45.573 | .000 |
| arousal 3 | 150 | 58.200 | .000 |
| arousal 5 | 150 | 83.440 | .000 |
| arousal 9 | 150 | 10.467 | .000 |
| arousal 10 | 150 | 70.427 | .000 |

Relaxation

| | | | |
|---|---|---|---|
| relaxation 1 | 150 | 131.120 | .000 |
| relaxation 2 | 150 | 163.227 | .000 |
| relaxation 5 | 150 | 80.720 | .000 |
| relaxation 6 | 150 | 11.640 | .000 |
| relaxation 7 | 150 | 82.587 | .000 |
| relaxation 10 | 150 | 228.933 | .000 |

Positive

| | | | |
|---|---|---|---|
| positive 2 | 150 | 35.040 | .000 |
| positive 3 | 150 | 90.533 | .000 |
| positive 4 | 150 | 101.920 | .000 |
| positive 5 | 150 | 66.040 | .000 |
| positive 7 | 150 | 143.813 | .000 |
| positive 8 | 150 | 128.027 | .000 |
| positive 9 | 150 | 47.013 | .000 |
| positive 10 | 150 | 138.053 | .000 |

Negative

| | | | |
|---|---|---|---|
| negative 1 | 150 | 119.920 | .000 |
| negative 2 | 150 | 59.440 | .000 |
| negative 5 | 150 | 117.360 | .000 |
| negative 9 | 150 | 62.080 | .000 |

Resurveys of the sound stimuli were conducted for relation to each emotion from the 150 subjects by using a seven-point scale based on 1 indicating strong disagreement to 7 indicating strong agreement.

Valid sounds relating to each emotion were analyzed using principal component analysis (PCA) based on Varimax (orthogonal) rotation. The analysis yielded four factors explaining of the variance for the entire set of variables. After obtaining the analysis result, representative sound stimuli for each emotion were derived, as shown in Table 2.

In Table 2, the bold type is the same factor, the italic character is the communalities <0.5, and the thick, underlined lettering represents the representative acoustic stimulus for each emotion.

TABLE 2

Component

| | 1 | 2 | 3 | 4 |
|---|---|---|---|---|
| positive 9 | .812 | .065 | .021 | −.033 |
| arousal 9 | .751 | −.353 | −.157 | .107 |
| relaxation 7 | .717 | .355 | .084 | .133 |
| positive 2 | .531 | −.202 | .203 | .107 |
| positive 3 | −.528 | .222 | .406 | −.003 |
| positive 8 | .520 | .142 | .161 | .074 |
| relaxation 2 | .192 | .684 | .109 | .004 |
| relaxation 1 | .028 | .649 | .168 | −.147 |
| relaxation 5 | −.290 | .629 | −.008 | .132 |
| relaxation 6 | .025 | .628 | −.061 | .107 |
| relaxation 10 | .052 | .569 | −.320 | −.187 |
| arousal 10 | −.201 | .529 | −.111 | .409 |
| positive 10 | −.145 | [symbol] | .342 | −.020 |
| negative 1 | −.257 | −.009 | .672 | .123 |
| positive 4 | .111 | .096 | .608 | −.185 |
| negative 2 | −.503 | .108 | .580 | .104 |
| negative 9 | .289 | −.252 | .566 | −.051 |
| negative 5 | .216 | −.232 | .528 | −.094 |
| positive 5 | .377 | .014 | [symbol] | −.019 |
| positive 7 | .002 | .193 | [symbol] | .128 |
| arousal 1 | −.158 | .209 | −.042 | .774 |
| arousal 2 | .129 | −.049 | .015 | .765 |
| arousal 5 | .210 | −.043 | .097 | .672 |
| arousal 3 | .566 | −.159 | −.140 | .617 |

### Experimental Procedure

Seventy undergraduate volunteers of both genders, evenly split between males and females, ranging in age from 20 to 30 years old with a mean of 24.52 years±0.64 years participated in this experiment. All subjects had normal or corrected-to-normal vision (i.e., over 0.8), and no family or medical history of disease involving visual function, cardiovascular system, or the central nervous system. Informed written consent was obtained from each subject prior to the study. This experimental study was approved by the Institutional Review Board of Sangmyung University, Seoul, South Korea (2015-8-1).

The experiment was composed of two trials where each trail was conducted for a duration of 5 minutes. The first trail was based on the motionlessness condition (MNC), which involves not moving or speaking. The second trial was based on a natural movement condition (NMC) involving simple conversations and slight movements. Participants repeatedly conducted the two trials and the order was randomized across the subjects. In order to verify the difference of movement between the two conditions, this experiment quantitatively measured the amount of movement during the experiment by using webcam images of each subject. In the present invention, the moving image may include at least one pupil, that is, one pupil or both pupils image.

The images were recorded at 30 frames per second (fps) with a resolution of 1920×1080 by using a HD Pro C920 camera from Logitech Inc. The movement measured the upper body and face based on MPEG-4 (Tekalp and Ostermann, 2000; JPandzic and Forchheimer, 2002). The movement in the upper body was extracted from the whole image based on frame differences. The upper body line was not tracking because the background was stationary.

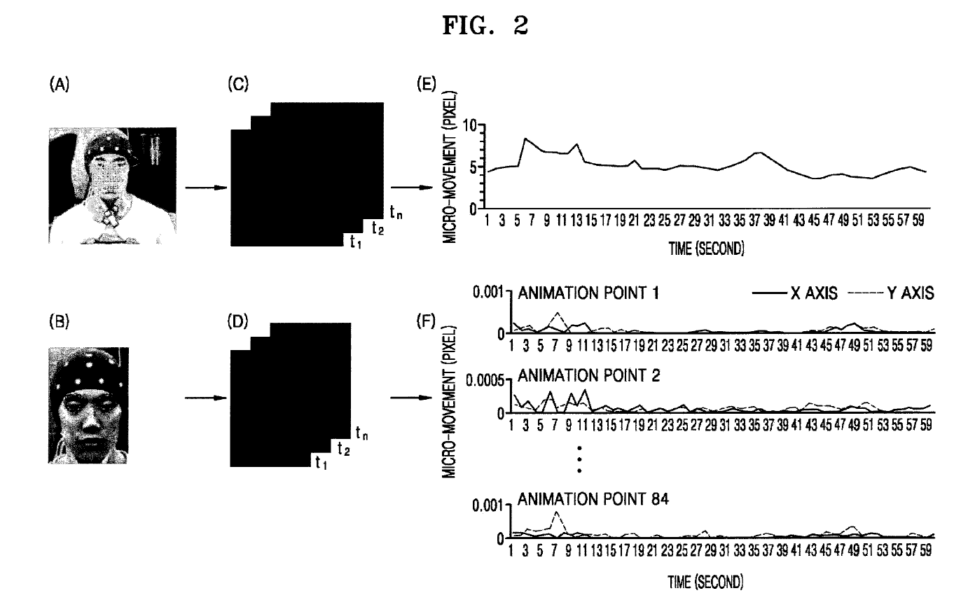

The movement in the face was extracted from 84 MPEG-4 animation points based on frame differences by using visage SDK 7.4 software from Visage Technologies Inc. All movement data used the mean value from each subject during the experiment and was compared to the difference of movement between the two trails, as shown in FIG. 2.

FIG. 2 shows an example of measuring the amount of motion of the subject's upper body in a state of the face is located at the intersection of the X axis and the Y axis,

In FIG. 2, (A) is an upper body image, (B) is a tracked face image at 84 MPEG-4 animation points, (C) and (D) shows the difference between before and after frames, (E) is a movement signal from the upper body, and (F) shows movement signals from 84 MPEG-4 animation points.

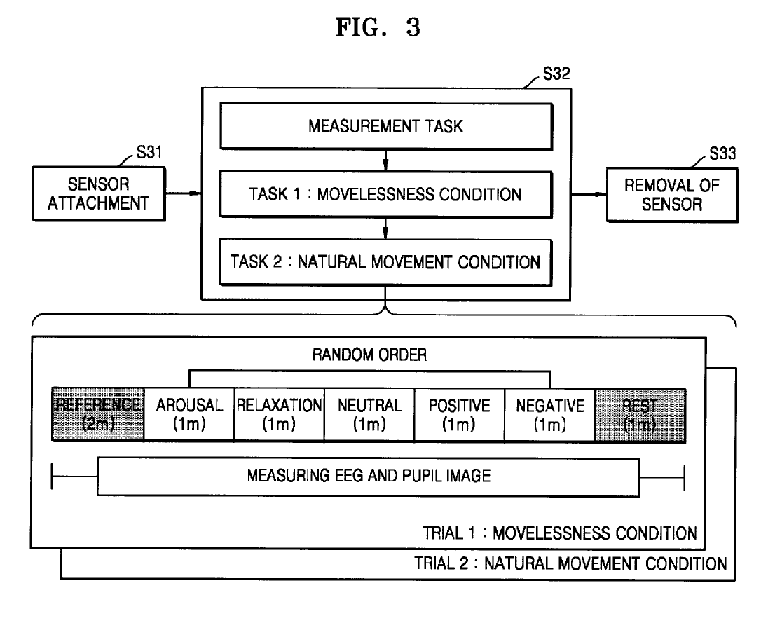

In order to cause the variation of physiological states, sound stimuli were presented to the participants during the trails. Each sound stimulus was randomly presented for 1 min for a total of five stimuli over the 5 min trial. A reference stimulus was presented for 3 min prior to the initiation of the task. The detailed experimental procedure is shown in FIG. 3.

The experimental procedure includes the sensor attachment S31, the measurement task S32 and the sensor removal S33 as shown in FIG. 3, and the measurement task S32 proceed as follows.

The experiment was conducted indoors with varying illumination caused by sunlight entering through the windows. The participants gazed at a black wall at a distance of 1.5 m while sitting in a comfortable chair. Sound stimuli were equally presented in both the trials by using earphones. The subjects were asked to constrict their movements and speaking during the MNC trial. However, the NMC trial involved a simple conversation and slight movement by the subjects. The subjects were asked to introduce themselves to another person as part of the conversation for sound stimuli thereby involving feelings and thinking of the sound stimuli. During the experiment, ECG signals and pupil image data were obtained.

ECG signals were sampled and recorded at a 500 Hz sampling rate through one channel with the lead-I method by an amplifier system including ECG 100C amplifiers and a MP100 power supply from BIOPAC System Inc. The ECG signals were digitized by a NI-DAQ-Pad 9205 of National Instrument Inc.

Pupil images were recorded at 125 fps with a resolution of 960×400 by GS3-U3-23S6M-C infrared camera from Point Grey Research Inc.

Hereinafter, a method for extracting or constructing (recovering) vital signs from a pupillary response will be described.

### Extraction of a Pupillary Response

The pupil detection procedure acquires moving images using the infrared video camera system as shown in FIG. 12, and then requires a specific image processing procedure

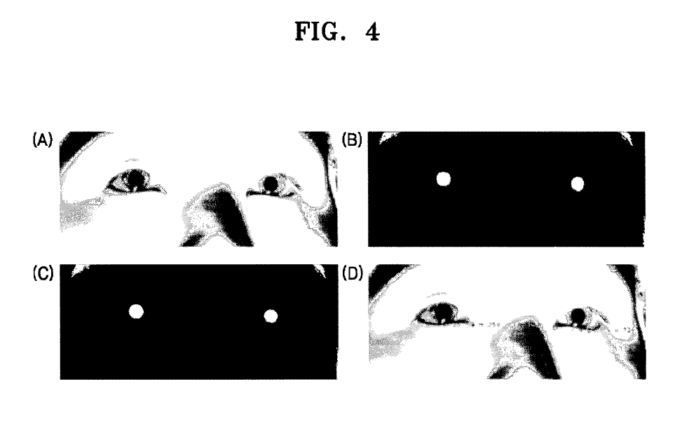

The pupil detection procedure may require following certain image processing steps since the images were captured using an infrared video camera, as shown in FIG. 4.

FIG. 4 shows a process of detecting a pupil region from the face image of a subject. In FIG. 4, (A) shows an input image (gray scale) obtained from a subject, (B) shows a binarized image based on an auto threshold, (C) shows pupil positions by the circular edge detection, and (D) shows the real-time detection result of the pupil region including the information about the center coordinates and the diameter of the pupil region. The threshold value was defined by a linear regression model that used a brightness value of the whole image, as shown in Equation 1.

Threshold=(−0.418×Bₘₑₐₙ+1.051×Bₘₐₓ)+7.973

B=Brightness value <Equation 1>

The next step to determine the pupil position involved processing the binary image by using a circular edge detection algorithm, as shown in Equation 2 (Daugman, 2004; Lee et al., 2009).

$${Max}_{( r , {x}_{0} , {y}_{0} )} \vert G σ ( r ) * \frac{δ}{δ r} {∮}_{r , {x}_{0} , {y}_{0}} \frac{I ( x , y )}{2 π r} ds \vert \\ I ( x , y ) = a grey level at the ( x , y ) position \\ ( {x}_{0} , {y}_{0} ) = center position of pupil \\ r = radius of pupil \qquad < Equation 2 >$$

In case that multiple pupil positions are selected, the reflected light caused by the infrared lamp may be used. Then an accurate pupil position was obtained, including centroid coordinates (x, y) and a diameter.

Pupil diameter data (signal) as a PSV was resampled at a frequency range of 1 Hz-30 Hz, as shown in Equation 3. The resampling procedure for the pupil diameter data involved a sampling rate of 30 data points, which then calculated the mean value during 1-s intervals by using a common sliding moving average technique (i.e., a window size of 1 second and a resolution of 1 second). However, non-tracked pupil diameter data caused by the eye closing was not involved in the resampling procedure.

$${( {SMA}_{m} )}_{x + n} = {( \frac{{\sum }_{i = 1}^{m} {P}_{i}}{m} )}_{x} , {( \frac{{\sum }_{i = 1}^{m} {P}_{i}}{m} )}_{x + 1} , … , {( \frac{{\sum }_{i = 1}^{m} {P}_{i}}{m} )}_{x + n} \\ SMA = sliding moving average \\ P = pupil diameter \qquad < Equation 3 >$$

### Detecting Frequency-Domain Index in Cardiac Activity

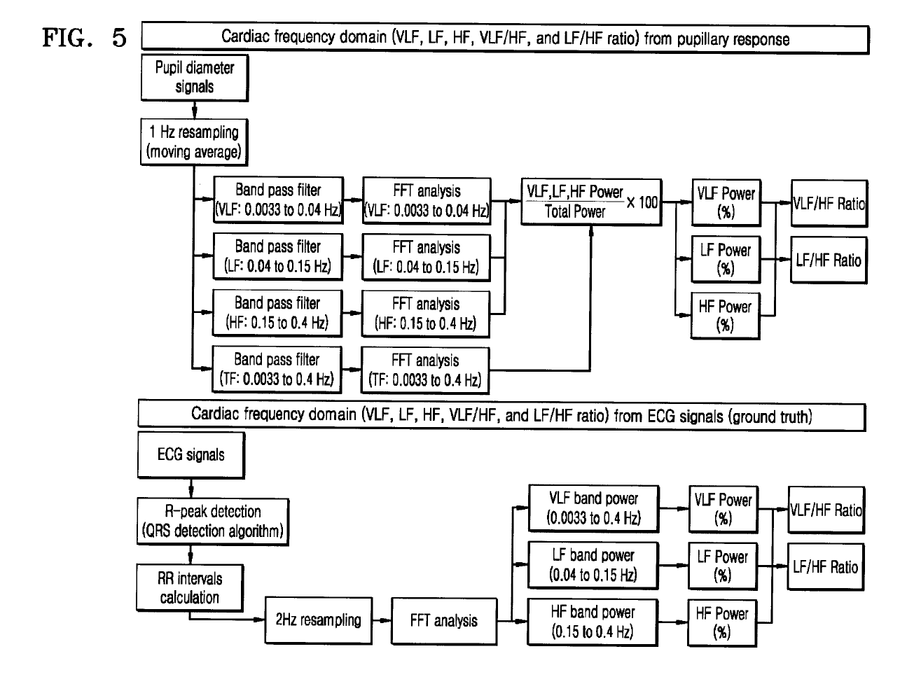

The detections of the cardiac frequency-domain indexes (parameters) are now described along with FIG. 5. Referring FIG. 5, the parameters of cardiac frequency-domain include heart rate variability (HRV) indexes which are determined and obtained from the pupillary response, and ECG signals (ground truth).

The HRV indexes, such as very low frequency (VLF), low frequency (LF), high frequency (HF), VLF/HF ratio, and LF/HF ratio, are the indicators of autonomic balance. The VLF band is an index of sympathetic activity while the HF band is an index of parasympathetic activity. The LF band is a complex mixture comprising of sympathetic and parasympathetic efferent and afferent activities as well as vascular system resonance (Malik, 1996; Shen et al., 2003; Reyes del Paso et al., 2013; Park of al., 2014).

Referring FIG. 5, the resampled pupil diameter data at 1 Hz was processed using a BPF of a VLF, LF, HF, and total frequency (TF) ranges, as shown in Equation (4). Each BPF band was applied by an HRV index frequency such as a VLF range of 0.0033 Hz-0.04 Hz; a LF range of 0.04 Hz-0.15 Hz; a HF range of 0.15 Hz-0.4 Hz; and a TF range of 0.0033 Hz-0.4 Hz (Malik, 1996; McCraty et al., 2009; Park et al., 2014). The filtered signals were extracted from the total power for each frequency band by using FFT analysis (i.e., the Hanning window technique).

$${X}_{k} = {\sum }_{n = 0}^{N - 1} {x}_{n} {e}^{- i 2 π k \frac{n}{N}} \\ k = 0 , … , N - 1 \qquad < Equation 4 >$$

The VLF, LF, and HF power were calculated from the ratio between total band power and the HRV index band power (VLF, LF, and HF), as shown in Equation (5). This procedure was processed by sliding window technique (window size: 180 sec and resolution: 1 sec).

$$VLF \text{/} LF \text{/} HF Power ( \% ) = \frac{VLF \text{/} LF \text{/} HF band power}{Total band power} \times 100 \qquad < Equation 5 >$$

The ECG signals detected the R-peak and then calculated the RRI. The successive RRI values were resampled at 2 Hz and analyzed to the HRV spectrum by using FFT, as shown in Equation (7). The HRV spectrum was categorized into frequency bands for a VLF band ranging from 0.0033 Hz to 0.04 Hz; a LF band ranging from 0.04 Hz to 0.15 Hz; and a HF band ranging from 0.15 Hz to 0.4 Hz. Then, power for each frequency band was extracted. The VLF, LF, and HF were converted into activity ratio such as VLF/HF or LF/HF ratio. The detailed procedure for processing ECG signals is shown in FIG. 5 showing a procedure of signal processing of cardiac frequency index.

### Result

The pupillary response was processed to extract the vital signs from the cardiac time domain index, cardiac frequency domain index, EEG spectral index, and the HEP index of the test subjects. These components were compared with each index from the sensor signals (i.e., ground truth) based on correlation coefficient (r) and mean error value (ME). The data was analyzed with respect both MNC and NMC for the test subjects.

To verify the difference of the amount movement between the two conditions of MNC and NMC, the movement data was quantitatively analyzed. The movement data was a normal distribution based on a normality test of probability-value (p)>0.05, and from an independent t-test. A Bonferroni correction was performed for the derived statistical significances (Dunnett, 1955). The statistical significance level was controlled based on the number of each individual hypothesis (i.e., α=0.05/n). The statistical significant level of the movement data sat up 0.0167 (upper body, X and Y axis in face, α=0.05/3). The effect size based on Cohen's d was also calculated to confirm practical significance. In Cohen's d, standard values of 0.10, 0.25, and 0.40 for effect size are generally regarded as small, medium, and large, respectively (Cohen, 2013).

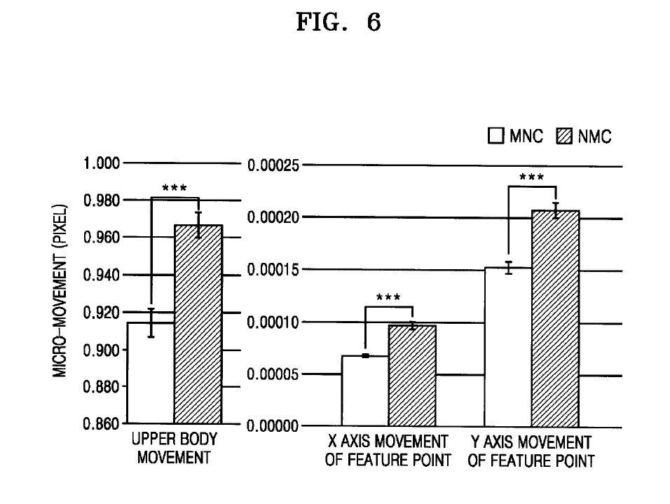

FIG. 6 shows averages of amount movement in upper body, X and Y axis in face for MNC and NMC (n=140, *** p<0.001) of one subject. Table 3 shows all subjects data of amount movement in upper body, X and Y axis in face for MNC and NMC.

Referring FIG. 6 and Table 3 according to the analysis, the amount of movement in MNC (upper body, X and Y axis for the face) are significantly increased compared to the NMC for the upper body (4138)=−5.121, p=0.000, Cohen's d=1.366 with large effect size), X axis for the face (4138)=−6.801, p=0.000, Cohen's d=1.158 with large effect size), and Y axis for the face (4138)=−6.255, p=0.000, Cohen's d=1.118 with large effect size).

TABLE 3

| | Subjects | | | | | |
|---|---|---|---|---|---|---|
| | Motionlessness Condition (MNC) | | | Natural Movement Condition (NMC) | | |
| | Upper | | | Upper | | |
| Subjects | body | X axis | Y axis | body | X axis | Y axis |
| S1 | 0.972675 | 0.000073 | 0.000158 | 1.003305 | 0.000117 | 0.000237 |
| S2 | 0.961020 | 0.000081 | 0.000170 | 1.002237 | 0.000101 | 0.000243 |
| S3 | 0.942111 | 0.000071 | 0.000206 | 0.945477 | 0.000081 | 0.000220 |
| S4 | 0.955444 | 0.000067 | 0.000189 | 0.960506 | 0.000072 | 0.000191 |
| S5 | 0.931979 | 0.000056 | 0.000106 | 0.972033 | 0.000070 | 0.000153 |
| S6 | 0.910416 | 0.000057 | 0.000103 | 0.999692 | 0.000086 | 0.000174 |
| S7 | 0.862268 | 0.000055 | 0.000216 | 0.867949 | 0.000071 | 0.000249 |
| S8 | 0.832109 | 0.000056 | 0.000182 | 0.884868 | 0.000068 | 0.000277 |
| S9 | 0.890771 | 0.000099 | 0.000188 | 0.890783 | 0.000099 | 0.000242 |
| S10 | 0.869373 | 0.000073 | 0.000168 | 0.872451 | 0.000089 | 0.000206 |
| S11 | 0.908724 | 0.000057 | 0.000128 | 0.963280 | 0.000102 | 0.000187 |
| S12 | 0.954168 | 0.000091 | 0.000180 | 0.964322 | 0.000181 | 0.000190 |
| S13 | 0.846164 | 0.000070 | 0.000144 | 0.917798 | 0.000079 | 0.000172 |
| S14 | 0.953219 | 0.000062 | 0.000116 | 1.024050 | 0.000093 | 0.000185 |
| S15 | 0.936300 | 0.000068 | 0.000202 | 0.952505 | 0.000101 | 0.000287 |
| S16 | 0.943040 | 0.000077 | 0.000220 | 0.958412 | 0.000106 | 0.000308 |
| S17 | 0.852292 | 0.000099 | 0.000199 | 0.901039 | 0.000077 | 0.000310 |
| S18 | 0.901182 | 0.000082 | 0.000278 | 0.920493 | 0.000084 | 0.000262 |
| S19 | 0.943810 | 0.000075 | 0.000156 | 0.974675 | 0.000099 | 0.000386 |
| S20 | 0.988983 | 0.000070 | 0.000162 | 1.029716 | 0.000175 | 0.000184 |
| S21 | 0.952451 | 0.000065 | 0.000102 | 1.005191 | 0.000081 | 0.000141 |
| S22 | 0.965017 | 0.000064 | 0.000099 | 0.999090 | 0.000183 | 0.000150 |
| S23 | 1.068848 | 0.000101 | 0.000200 | 1.090858 | 0.000108 | 0.000255 |
| S24 | 0.993841 | 0.000092 | 0.000184 | 1.052424 | 0.000111 | 0.000247 |
| S25 | 0.883615 | 0.000064 | 0.000258 | 0.913927 | 0.000077 | 0.000283 |
| S26 | 0.870531 | 0.000051 | 0.000221 | 0.906540 | 0.000074 | 0.000252 |
| S27 | 0.955718 | 0.000064 | 0.000126 | 0.963460 | 0.000071 | 0.000169 |
| S28 | 0.968524 | 0.000061 | 0.000142 | 0.985782 | 0.000075 | 0.000184 |
| S29 | 0.794718 | 0.000067 | 0.000119 | 0.918873 | 0.000074 | 0.000136 |
| S30 | 0.817818 | 0.000064 | 0.000105 | 0.914591 | 0.000073 | 0.000148 |
| S31 | 0.937005 | 0.000053 | 0.000138 | 0.979654 | 0.000080 | 0.000203 |
| S32 | 0.974895 | 0.000067 | 0.000204 | 1.011137 | 0.000072 | 0.000215 |
| S33 | 0.877308 | 0.000073 | 0.000134 | 0.899194 | 0.000087 | 0.000196 |
| S34 | 0.867672 | 0.000063 | 0.000127 | 0.894298 | 0.000077 | 0.000188 |
| S35 | 0.948874 | 0.000099 | 0.000182 | 0.952532 | 0.000105 | 0.000217 |
| S36 | 0.968912 | 0.000109 | 0.000217 | 1.020322 | 0.000115 | 0.000240 |
| S37 | 0.811181 | 0.000063 | 0.000204 | 0.964774 | 0.000071 | 0.000244 |
| S38 | 0.921204 | 0.000061 | 0.000160 | 0.966262 | 0.000071 | 0.000213 |
| S39 | 0.907618 | 0.000060 | 0.000151 | 0.951832 | 0.000076 | 0.000188 |
| S40 | 0.907953 | 0.000061 | 0.000169 | 0.920784 | 0.000071 | 0.000188 |
| S41 | 0.907145 | 0.000055 | 0.000151 | 0.937417 | 0.000171 | 0.000196 |
| S42 | 0.909996 | 0.000055 | 0.000163 | 0.995645 | 0.000072 | 0.000222 |
| S43 | 0.940886 | 0.000061 | 0.000137 | 0.971473 | 0.000082 | 0.000188 |
| S44 | 0.979163 | 0.000059 | 0.000127 | 1.058006 | 0.000184 | 0.000244 |
| S45 | 0.946343 | 0.000056 | 0.000109 | 1.029439 | 0.000082 | 0.000156 |
| S46 | 0.951810 | 0.000061 | 0.000154 | 0.977621 | 0.000087 | 0.000256 |
| S47 | 0.809073 | 0.000060 | 0.000147 | 0.961375 | 0.000065 | 0.000252 |
| S48 | 0.961124 | 0.000073 | 0.000176 | 0.997457 | 0.000083 | 0.000189 |
| S49 | 0.994281 | 0.000074 | 0.000172 | 1.020115 | 0.000094 | 0.000222 |
| S50 | 0.853841 | 0.000075 | 0.000194 | 0.978026 | 0.000104 | 0.000247 |
| S51 | 0.818171 | 0.000059 | 0.000168 | 0.850567 | 0.000091 | 0.000255 |
| S52 | 0.845488 | 0.000072 | 0.000134 | 0.895100 | 0.000105 | 0.000293 |
| S53 | 0.899975 | 0.000081 | 0.000150 | 0.967366 | 0.000094 | 0.000179 |
| S54 | 0.819878 | 0.000057 | 0.000106 | 0.907099 | 0.000108 | 0.000193 |
| S55 | 0.824809 | 0.000061 | 0.000119 | 0.854062 | 0.000062 | 0.000125 |
| S56 | 0.829834 | 0.000067 | 0.000126 | 0.915019 | 0.000169 | 0.000157 |
| S57 | 0.836302 | 0.000066 | 0.000126 | 0.892036 | 0.000083 | 0.000172 |
| S58 | 0.876029 | 0.000065 | 0.000155 | 0.988827 | 0.000186 | 0.000163 |
| S59 | 0.876581 | 0.000065 | 0.000149 | 0.924143 | 0.000117 | 0.000296 |
| S60 | 0.881068 | 0.000101 | 0.000252 | 1.063924 | 0.000109 | 0.000381 |
| S61 | 0.880455 | 0.000055 | 0.000093 | 1.007333 | 0.000080 | 0.000190 |
| S62 | 0.900065 | 0.000055 | 0.000087 | 1.028052 | 0.000076 | 0.000176 |
| S63 | 1.045809 | 0.000056 | 0.000102 | 1.061254 | 0.000096 | 0.000161 |
| S64 | 1.067929 | 0.000052 | 0.000105 | 1.070771 | 0.000111 | 0.000162 |
| S65 | 0.949971 | 0.000055 | 0.000101 | 1.004960 | 0.000068 | 0.000143 |
| S66 | 0.964054 | 0.000053 | 0.000093 | 1.068673 | 0.000169 | 0.000140 |
| S67 | 0.828268 | 0.000054 | 0.000082 | 0.886462 | 0.000061 | 0.000117 |
| S68 | 0.922679 | 0.000049 | 0.000079 | 0.945291 | 0.000061 | 0.000102 |
| S69 | 0.946723 | 0.000063 | 0.000112 | 1.069926 | 0.000114 | 0.000119 |
| S70 | 0.977655 | 0.000064 | 0.000113 | 0.999438 | 0.000065 | 0.000119 |
| mean | 0.914217 | 0.000067 | 0.000153 | 0.966343 | 0.000096 | 0.000208 |
| SD | 0.061596 | 0.000014 | 0.000044 | 0.057911 | 0.000033 | 0.000058 |

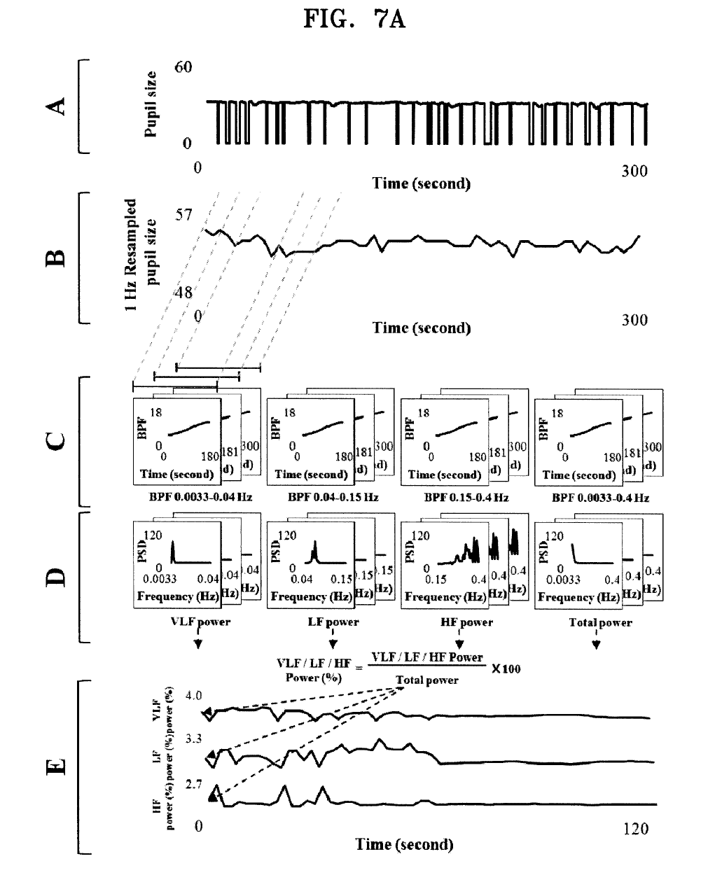

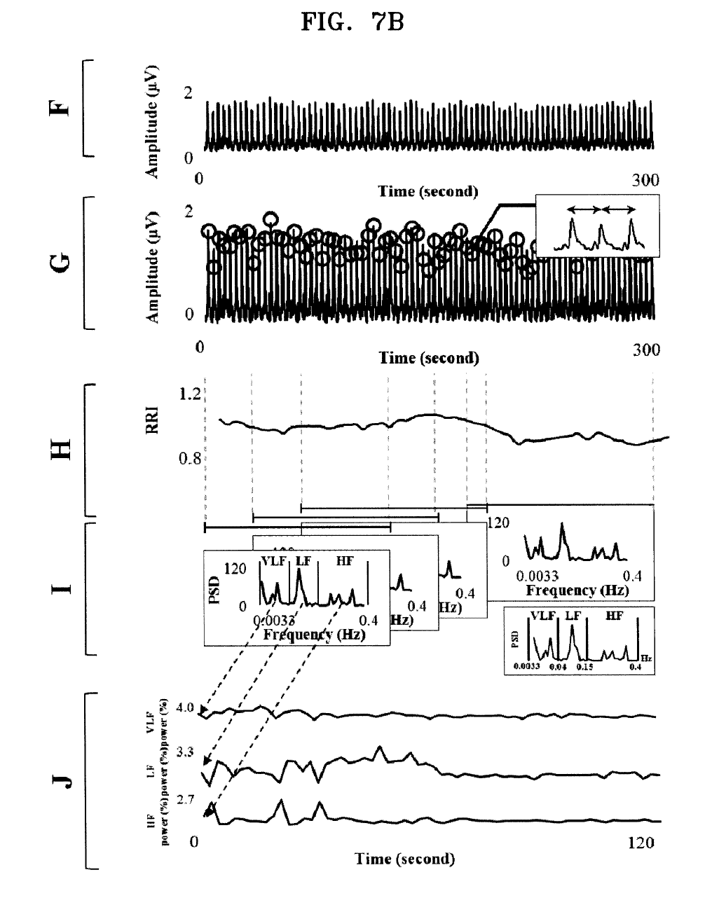

The cardiac frequency domain indexes, such as VLF power, LF power, HF power, VLF/HF ratio, and LF/HF ratio, for the cardiac output were extracted from the pupillary response. These components were compared with the frequency index from the ECG signals (ground truth). FIGS. 7A and 7B show examples of detecting and extracting the HRV indexes from the pupillary response and ECG signals.

This experiment was able to determine the cardiac frequency index (i.e., VLF power, LF power, HF power, VLF/HF power ratio, and LF/HF power ratio) from the pupillary response by the entrainment of the harmonic frequency.

The cardiac HRV index range of 0.0033 Hz-0.4 Hz was closely connected with the circadian pupillary rhythm of the same frequency range. The size variation of the pupil diameter was divided into three bands: VLF (0.0033 Hz-0.04 Hz), LF (0.04 Hz-0.15 Hz), and HF (0.15 Hz-0.4 Hz).

In addition, the size variation was extracted for each power band from the ratio between the total power bands (0.0033 Hz-0.4 Hz). These were synchronized with the HRV index within the frequency band of 1 Hz. The VLF/HF and LF/HF power ratio were then calculated from the individual VLF, LF, and HF components.

In FIG. 7, (A) shows frame difference signals of pupil size, (B) shows a wave form by signals of 1 Hz resampled based on sliding moving average (window size: 30 fps and resolution: 30 fps), (C) shows band pass filtered signals of each frequency band (VLF: 0.0033 Hz-0.04 Hz, LF: 0.04 Hz-0.15 Hz, HF: 0.15 Hz-0.4 Hz, and TF: 0.0033 Hz-0.4 Hz), (D) shows signals of FFT analysis, (E) shows signals of VLF, LF, and HF power calculated from the proportion with TF power (pupillary response), (F) shows an example of ECG raw signals. (G) shows detecting the R-peak (QRS complex) and R-peak to R-peak intervals, (H) show heart rate (RRI) signals, (I) shows heart rate variability (HRV) analysis and extraction of VLF, LF, and HF power, and (J) shows VLF, LF, and HF power signals from ECG signals (ground truth).

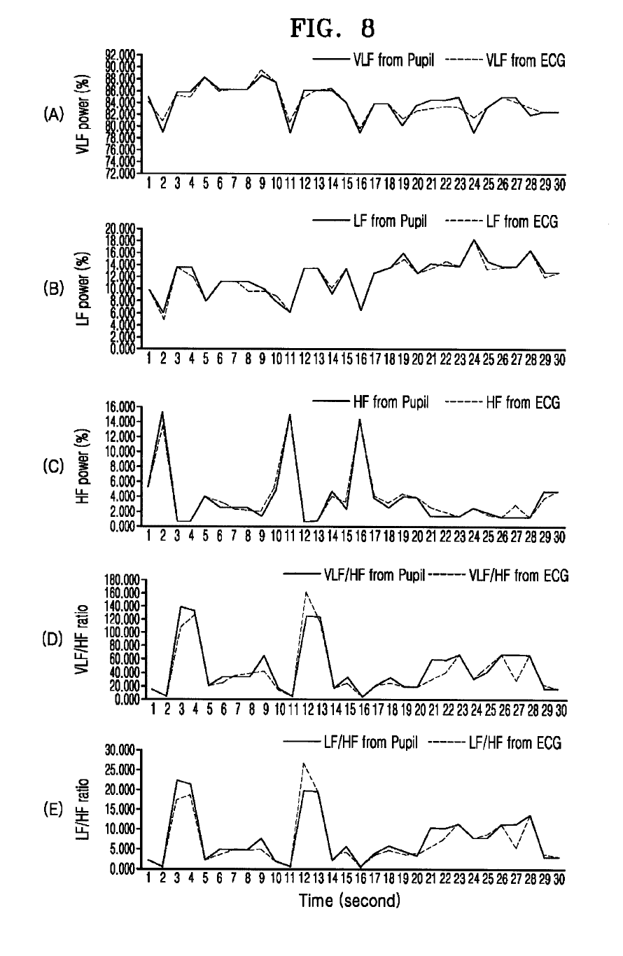

FIG. 8 shows an example of the cardiac frequency index extracted from the pupillary response and ECG signals of a subject in MNC, where r=0.940, ME=0.009 for VLF, r=0.980, ME=0.179 for LF, r=0.989, ME=0.091 for HF, r=0.938, ME=3.902 for VLF/HF ratio, r=0.937, ME=0.669 for LF/HF ratio.

Table 4 shows average of correlation coefficient and mean error of cardiac frequency index in MNC (N=270, p<0.01). These results was processed by using the sliding window technique based on a window size of 180 sec and a resolution of 1 sec by using the recorded data for 300 sec. The correlation and mean error were the mean value for the 70 subjects (in one subject, N=120).

TABLE 4

| | Correlation coefficient | | | | | Mean error | | | | |
|---|---|---|---|---|---|---|---|---|---|---|
| Subjects | VLF | LF | HF | VLF/HF | LF/HF | VLF | LF | HF | VLF/HF | LF/HF |
| S1 | 0.855 | 0.819 | 0.947 | 0.728 | 0.786 | 0.252 | 0.034 | 0.225 | 0.340 | 0.308 |
| S2 | 0.836 | 0.944 | 0.930 | 0.615 | 0.686 | 0.511 | 0.208 | 0.024 | 0.562 | 0.700 |
| S3 | 0.918 | 0.957 | 0.815 | 0.872 | 0.727 | 0.075 | 0.178 | 0.361 | 0.174 | 0.282 |
| S4 | 0.950 | 0.901 | 0.914 | 0.810 | 0.770 | 0.083 | 0.031 | 0.043 | 0.173 | 0.144 |
| S5 | 0.929 | 0.978 | 0.967 | 0.714 | 0.731 | 0.013 | 0.042 | 0.083 | 0.149 | 0.135 |
| S6 | 0.835 | 0.887 | 0.972 | 0.731 | 0.811 | 0.301 | 0.501 | 0.482 | 1.453 | 1.353 |
| S7 | 0.905 | 0.924 | 0.888 | 0.843 | 0.794 | 0.312 | 0.628 | 0.592 | 0.444 | 0.459 |
| S8 | 0.845 | 0.955 | 0.937 | 0.813 | 0.709 | 0.193 | 0.642 | 0.428 | 0.635 | 0.767 |
| S9 | 0.943 | 0.854 | 0.820 | 0.764 | 0.946 | 0.192 | 0.151 | 0.104 | 0.862 | 0.697 |
| S10 | 0.779 | 0.714 | 0.717 | 0.630 | 0.656 | 1.693 | 1.369 | 1.134 | 1.819 | 1.703 |
| S11 | 0.884 | 0.852 | 0.844 | 0.820 | 0.859 | 0.609 | 0.334 | 0.382 | 0.786 | 0.735 |
| S12 | 0.878 | 0.850 | 0.893 | 0.702 | 0.853 | 0.239 | 0.429 | 0.417 | 0.284 | 0.286 |
| S13 | 0.861 | 0.871 | 0.819 | 0.710 | 0.751 | 0.323 | 0.436 | 0.373 | 0.390 | 0.300 |
| S14 | 0.876 | 0.923 | 0.873 | 0.709 | 0.907 | 0.689 | 0.534 | 0.101 | 0.332 | 0.352 |
| S15 | 0.929 | 0.894 | 0.886 | 0.790 | 0.785 | 0.263 | 0.265 | 0.212 | 0.129 | 0.125 |
| S16 | 0.875 | 0.880 | 0.909 | 0.795 | 0.915 | 0.220 | 0.316 | 0.551 | 0.734 | 0.743 |
| S17 | 0.903 | 0.891 | 0.900 | 0.842 | 0.946 | 0.648 | 0.599 | 0.350 | 0.168 | 0.179 |
| S18 | 0.896 | 0.908 | 0.911 | 0.713 | 0.705 | 0.326 | 0.378 | 0.269 | 0.648 | 0.650 |
| S19 | 0.832 | 0.963 | 0.898 | 0.883 | 0.955 | 0.430 | 0.398 | 0.104 | 1.288 | 1.126 |
| S20 | 0.911 | 0.951 | 0.944 | 0.752 | 0.802 | 0.342 | 0.670 | 0.194 | 0.140 | 0.157 |
| S21 | 0.893 | 0.980 | 0.915 | 0.825 | 0.808 | 0.340 | 0.272 | 0.275 | 0.717 | 0.684 |
| S22 | 0.944 | 0.871 | 0.976 | 0.802 | 0.917 | 0.092 | 0.087 | 0.114 | 0.669 | 0.665 |
| S23 | 0.917 | 0.980 | 0.968 | 0.808 | 0.700 | 0.320 | 0.139 | 0.321 | 0.760 | 0.729 |
| S24 | 0.881 | 0.822 | 0.971 | 0.902 | 0.902 | 0.348 | 0.425 | 0.522 | 0.565 | 0.531 |
| S25 | 0.832 | 0.876 | 0.846 | 0.721 | 0.702 | 0.572 | 0.215 | 0.589 | 1.442 | 1.343 |
| S26 | 0.898 | 0.932 | 0.914 | 0.827 | 0.783 | 0.686 | 0.344 | 0.202 | 0.390 | 0.344 |
| S27 | 0.855 | 0.918 | 0.885 | 0.713 | 0.776 | 0.576 | 0.012 | 0.289 | 0.492 | 0.583 |
| S28 | 0.863 | 0.876 | 0.932 | 0.889 | 0.766 | 0.115 | 0.247 | 0.242 | 0.550 | 0.651 |
| S29 | 0.849 | 0.839 | 0.830 | 0.806 | 0.843 | 0.215 | 0.120 | 0.586 | 0.421 | 0.345 |
| S30 | 0.894 | 0.972 | 0.981 | 0.924 | 0.936 | 0.209 | 0.059 | 0.084 | 0.372 | 0.311 |
| S31 | 0.940 | 0.980 | 0.989 | 0.938 | 0.937 | 0.009 | 0.179 | 0.091 | 0.102 | 0.069 |
| S32 | 0.908 | 0.933 | 0.821 | 0.746 | 0.777 | 0.336 | 0.414 | 0.198 | 1.096 | 0.997 |
| S33 | 0.917 | 0.856 | 0.938 | 0.859 | 0.850 | 0.671 | 0.450 | 0.446 | 0.271 | 0.341 |
| S34 | 0.908 | 0.845 | 0.882 | 0.835 | 0.904 | 0.059 | 0.204 | 0.143 | 0.706 | 0.735 |
| S35 | 0.940 | 0.980 | 0.989 | 0.938 | 0.937 | 0.009 | 0.179 | 0.091 | 0.026 | 0.069 |
| S36 | 0.849 | 0.835 | 0.822 | 0.899 | 0.841 | 0.229 | 0.654 | 0.393 | 0.559 | 0.516 |
| S37 | 0.920 | 0.855 | 0.832 | 0.785 | 0.712 | 0.042 | 0.358 | 0.025 | 0.153 | 0.103 |
| S38 | 0.939 | 0.818 | 0.874 | 0.822 | 0.939 | 0.105 | 0.154 | 0.026 | 0.025 | 0.052 |
| S39 | 0.873 | 0.927 | 0.931 | 0.850 | 0.705 | 0.454 | 0.089 | 0.493 | 0.612 | 0.682 |
| S40 | 0.924 | 0.835 | 0.890 | 0.898 | 0.764 | 0.230 | 0.038 | 0.436 | 0.054 | 0.054 |
| S41 | 0.937 | 0.910 | 0.851 | 0.804 | 0.758 | 0.165 | 0.151 | 0.151 | 0.293 | 0.276 |
| S42 | 0.888 | 0.872 | 0.910 | 0.841 | 0.891 | 0.378 | 0.114 | 0.412 | 0.597 | 0.636 |
| S43 | 0.834 | 0.914 | 0.829 | 0.868 | 0.792 | 0.396 | 0.660 | 0.547 | 0.632 | 0.629 |
| S44 | 0.831 | 0.866 | 0.888 | 0.751 | 0.905 | 0.631 | 0.697 | 0.109 | 0.663 | 0.643 |
| S45 | 0.933 | 0.845 | 0.955 | 0.854 | 0.749 | 0.230 | 0.221 | 0.330 | 0.372 | 0.228 |
| S46 | 0.836 | 0.894 | 0.881 | 0.734 | 0.775 | 0.461 | 0.555 | 0.205 | 0.246 | 0.209 |
| S47 | 0.847 | 0.956 | 0.842 | 0.898 | 0.771 | 0.210 | 0.413 | 0.435 | 0.687 | 0.666 |
| S48 | 0.902 | 0.817 | 0.856 | 0.726 | 0.682 | 0.484 | 0.118 | 0.236 | 0.237 | 0.205 |
| S49 | 0.923 | 0.940 | 0.901 | 0.874 | 0.739 | 0.633 | 0.245 | 0.610 | 0.187 | 0.134 |
| S50 | 0.856 | 0.867 | 0.926 | 0.742 | 0.886 | 0.376 | 0.482 | 0.129 | 0.610 | 0.617 |
| S51 | 0.923 | 0.888 | 0.836 | 0.836 | 0.709 | 0.014 | 0.211 | 0.305 | 0.234 | 0.242 |
| S52 | 0.878 | 0.965 | 0.837 | 0.918 | 0.729 | 0.598 | 0.406 | 0.110 | 0.248 | 0.246 |
| S53 | 0.882 | 0.915 | 0.836 | 0.707 | 0.789 | 0.299 | 0.658 | 0.054 | 0.682 | 0.621 |
| S54 | 0.844 | 0.896 | 0.950 | 0.795 | 0.737 | 0.694 | 0.109 | 0.039 | 0.536 | 0.556 |
| S55 | 0.832 | 0.938 | 0.956 | 0.715 | 0.843 | 0.230 | 0.351 | 0.256 | 1.473 | 1.297 |
| S56 | 0.909 | 0.918 | 0.870 | 0.802 | 0.919 | 0.507 | 0.470 | 0.511 | 0.552 | 0.577 |
| S57 | 0.831 | 0.832 | 0.934 | 0.885 | 0.777 | 0.215 | 0.165 | 0.046 | 0.655 | 0.661 |
| S58 | 0.915 | 0.926 | 0.964 | 0.682 | 0.644 | 0.321 | 0.123 | 0.585 | 0.090 | 0.035 |
| S59 | 0.917 | 0.969 | 0.873 | 0.715 | 0.712 | 0.589 | 0.565 | 0.581 | 0.167 | 0.171 |
| S60 | 0.834 | 0.859 | 0.971 | 0.865 | 0.753 | 0.468 | 0.982 | 1.450 | 0.925 | 0.970 |
| S61 | 0.903 | 0.975 | 0.844 | 0.827 | 0.815 | 0.564 | 0.094 | 0.061 | 0.609 | 0.639 |
| S62 | 0.740 | 0.723 | 0.842 | 0.610 | 0.696 | 0.403 | 0.230 | 0.547 | 1.219 | 1.493 |
| S63 | 0.974 | 0.982 | 0.911 | 0.937 | 0.982 | 0.664 | 0.214 | 0.450 | 0.020 | 0.013 |
| S64 | 0.971 | 0.940 | 0.915 | 0.742 | 0.718 | 0.174 | 0.181 | 0.191 | 0.474 | 0.336 |
| S65 | 0.946 | 0.901 | 0.899 | 0.725 | 0.738 | 0.055 | 0.046 | 0.097 | 0.148 | 0.550 |
| S66 | 0.873 | 0.967 | 0.825 | 0.761 | 0.743 | 0.016 | 0.412 | 0.087 | 0.108 | 0.103 |
| S67 | 0.918 | 0.971 | 0.949 | 0.910 | 0.870 | 0.587 | 0.651 | 0.296 | 0.099 | 0.081 |
| S68 | 0.930 | 0.845 | 0.920 | 0.736 | 0.853 | 0.227 | 0.168 | 0.107 | 0.011 | 0.044 |
| S69 | 0.867 | 0.828 | 0.900 | 0.721 | 0.695 | 0.189 | 0.341 | 0.057 | 0.181 | 0.216 |
| S70 | 0.920 | 0.870 | 0.899 | 0.793 | 0.821 | 0.596 | 0.249 | 0.079 | 0.344 | 0.329 |
| mean | 0.888 | 0.898 | 0.896 | 0.797 | 0.801 | 0.353 | 0.329 | 0.301 | 0.497 | 0.492 |
| SD | 0.044 | 0.058 | 0.054 | 0.080 | 0.086 | 0.258 | 0.243 | 0.250 | 0.386 | 0.372 |

In the comparison result with ground truth in MNC, the cardiac frequency index from pupillary response were strong correlation of all parameters with r=0.888±0.044 for VLF power; r=0.898±0.058 for LF power; r=0.896±0.054 for HF power; r=0.797±0.080 for VLF/HF ratio; and r=0.801±0.086 for LF/HF ratio, as shown Table 4.

Also, the difference between the mean error of all parameters was low with ME=0.353±0.258 for VLF power; ME=0.329±0.243 for LF power; ME=0.301±0.250 for HF power; ME=0.497±0.386 for VLF/HF ratio, and ME=0.492±0.372 for LF/HF ratio.

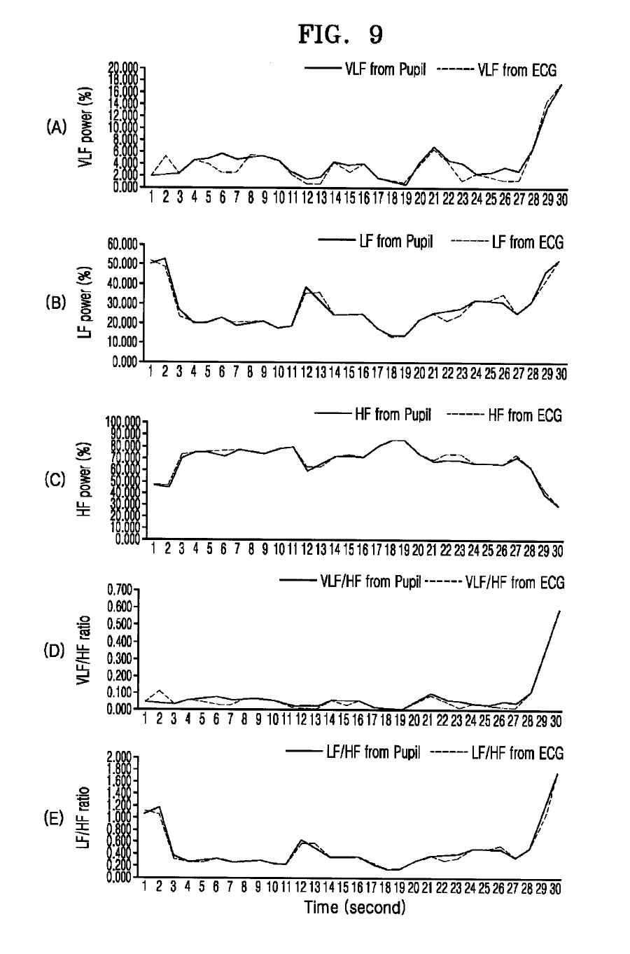

FIG. 9 shows an example of the cardiac frequency index extracted from the pupillary response and ECG signals of a subject in NMC, where r=0.945, ME=0.417 for VLF, r=0.983, ME=0.485 for LF, r=0.989, ME=0.935 for HF, r=0.985, ME=0.006 for VLF/HF ratio, r=0.990, and ME=0.016 for LF/HF ratio.

Table 5 shows an average of correlation coefficients and a mean error of the cardiac frequency index of the subjects in NMC (N=120, p<0.01). The procedure for Table 5 was performed by using the sliding window technique where the window size was 180 sec and the resolution size was 1 sec by using the recorded data for 300 s.

TABLE 5

| | Correlation coefficient | | | | | Mean error | | | | |
|---|---|---|---|---|---|---|---|---|---|---|
| Subjects | HR | BPM | SDNN | rMSSD | pNN50 | HR | BPM | SDNN | rMSSD | pNN50 |
| S1 | 0.760 | 0.760 | 0.590 | 0.940 | 0.819 | 0.014 | 1.631 | 5.792 | 0.165 | 1.411 |
| S2 | 0.819 | 0.819 | 0.707 | 0.958 | 0.846 | 0.009 | 1.093 | 3.647 | 0.109 | 1.615 |
| S3 | 0.923 | 0.923 | 0.680 | 0.765 | 0.797 | 0.007 | 0.958 | 5.027 | 0.324 | 3.179 |
| S4 | 0.780 | 0.780 | 0.658 | 0.955 | 0.626 | 0.006 | 0.886 | 2.761 | 0.121 | 1.026 |
| S5 | 0.801 | 0.801 | 0.646 | 0.906 | 0.805 | 0.005 | 0.807 | 2.802 | 0.061 | 1.248 |
| S6 | 0.680 | 0.680 | 0.807 | 0.906 | 0.738 | 0.011 | 1.167 | 1.872 | 0.057 | 2.157 |
| S7 | 0.583 | 0.583 | 0.665 | 0.980 | 0.737 | 0.008 | 0.880 | 7.492 | 0.097 | 2.735 |
| S8 | 0.948 | 0.948 | 0.631 | 0.875 | 0.688 | 0.008 | 0.831 | 6.002 | 0.054 | 2.482 |
| S9 | 0.874 | 0.874 | 0.770 | 0.990 | 0.636 | 0.009 | 0.963 | 3.763 | 0.065 | 4.957 |
| S10 | 0.744 | 0.744 | 0.593 | 0.929 | 0.684 | 0.014 | 1.275 | 2.677 | 0.178 | 2.962 |
| S11 | 0.929 | 0.929 | 0.662 | 0.989 | 0.833 | 0.014 | 1.221 | 3.819 | 0.149 | 1.599 |
| S12 | 0.951 | 0.951 | 0.936 | 0.994 | 0.765 | 0.025 | 1.438 | 7.297 | 0.365 | 2.057 |
| S13 | 0.664 | 0.664 | 0.531 | 0.897 | 0.801 | 0.026 | 1.553 | 4.627 | 0.368 | 3.030 |
| S14 | 0.816 | 0.816 | 0.777 | 0.971 | 0.642 | 0.015 | 1.258 | 3.700 | 0.047 | 3.348 |
| S15 | 0.795 | 0.795 | 0.786 | 0.988 | 0.797 | 0.019 | 1.583 | 3.513 | 0.049 | 2.664 |
| S16 | 0.890 | 0.890 | 0.911 | 0.984 | 0.634 | 0.007 | 0.936 | 1.510 | 0.187 | 2.986 |
| S17 | 0.915 | 0.915 | 0.617 | 0.983 | 0.798 | 0.007 | 0.937 | 5.137 | 0.183 | 1.681 |
| S18 | 0.787 | 0.787 | 0.937 | 0.977 | 0.868 | 0.017 | 1.337 | 1.045 | 0.096 | 2.625 |
| S19 | 0.779 | 0.779 | 0.886 | 0.983 | 0.602 | 0.015 | 1.094 | 1.497 | 0.087 | 3.138 |
| S20 | 0.915 | 0.917 | 0.559 | 0.713 | 0.620 | 0.007 | 0.934 | 8.762 | 0.590 | 3.138 |
| S21 | 0.932 | 0.932 | 0.674 | 0.897 | 0.836 | 0.006 | 0.919 | 7.140 | 0.206 | 2.134 |
| S22 | 0.929 | 0.929 | 0.813 | 0.974 | 0.805 | 0.006 | 0.866 | 3.100 | 0.088 | 1.427 |
| S23 | 0.634 | 0.634 | 0.638 | 0.954 | 0.662 | 0.033 | 3.858 | 2.985 | 0.215 | 0.717 |
| S24 | 0.822 | 0.822 | 0.894 | 0.995 | 0.710 | 0.017 | 1.819 | 1.458 | 0.123 | 3.229 |
| S25 | 0.842 | 0.842 | 0.754 | 0.985 | 0.754 | 0.016 | 1.749 | 2.211 | 0.062 | 1.108 |
| S26 | 0.871 | 0.871 | 0.637 | 0.993 | 0.638 | 0.010 | 1.118 | 3.722 | 0.195 | 2.399 |
| S27 | 0.822 | 0.822 | 0.658 | 0.976 | 0.694 | 0.025 | 1.377 | 6.905 | 0.101 | 2.765 |
| S28 | 0.751 | 0.751 | 0.661 | 0.765 | 0.796 | 0.016 | 0.949 | 3.741 | 0.226 | 0.267 |
| S29 | 0.948 | 0.948 | 0.683 | 0.976 | 0.625 | 0.007 | 1.192 | 2.867 | 0.175 | 0.884 |
| S30 | 0.876 | 0.876 | 0.796 | 0.976 | 0.628 | 0.005 | 0.751 | 4.662 | 0.170 | 1.679 |
| S31 | 0.850 | 0.850 | 0.642 | 0.743 | 0.847 | 0.015 | 1.351 | 1.505 | 0.163 | 2.675 |
| S32 | 0.878 | 0.878 | 0.814 | 0.992 | 0.708 | 0.012 | 1.154 | 3.122 | 0.204 | 0.982 |
| S33 | 0.945 | 0.945 | 0.816 | 0.971 | 0.853 | 0.013 | 1.013 | 2.415 | 0.112 | 1.925 |
| S34 | 0.737 | 0.737 | 0.630 | 0.976 | 0.730 | 0.015 | 1.416 | 2.769 | 0.076 | 0.847 |
| S35 | 0.812 | 0.812 | 0.622 | 0.966 | 0.816 | 0.016 | 1.396 | 4.748 | 0.059 | 1.924 |
| S36 | 0.714 | 0.714 | 0.622 | 0.745 | 0.627 | 0.010 | 1.208 | 2.266 | 0.174 | 0.919 |
| S37 | 0.828 | 0.828 | 0.626 | 0.839 | 0.607 | 0.009 | 1.085 | 2.464 | 0.252 | 1.644 |
| S38 | 0.747 | 0.747 | 0.645 | 0.758 | 0.793 | 0.020 | 2.090 | 7.520 | 0.138 | 2.080 |
| S39 | 0.869 | 0.869 | 0.665 | 0.984 | 0.635 | 0.012 | 1.190 | 1.077 | 0.139 | 1.272 |
| S40 | 0.684 | 0.684 | 0.601 | 0.987 | 0.809 | 0.010 | 1.134 | 1.239 | 0.068 | 2.917 |
| S41 | 0.733 | 0.733 | 0.802 | 0.814 | 0.769 | 0.013 | 1.623 | 4.872 | 0.149 | 2.989 |
| S42 | 0.702 | 0.702 | 0.603 | 0.732 | 0.774 | 0.009 | 1.217 | 2.570 | 0.308 | 0.333 |
| S43 | 0.875 | 0.875 | 0.613 | 0.930 | 0.668 | 0.025 | 3.156 | 1.341 | 0.261 | 2.728 |
| S44 | 0.691 | 0.691 | 0.686 | 0.817 | 0.773 | 0.023 | 2.469 | 2.685 | 0.533 | 0.985 |
| S45 | 0.855 | 0.855 | 0.868 | 0.998 | 0.847 | 0.012 | 1.230 | 2.694 | 0.068 | 0.909 |
| S46 | 0.883 | 0.883 | 0.841 | 0.984 | 0.749 | 0.020 | 2.460 | 4.043 | 0.065 | 1.895 |
| S47 | 0.964 | 0.964 | 0.788 | 0.986 | 0.693 | 0.008 | 0.986 | 10.771 | 0.205 | 0.140 |
| S48 | 0.821 | 0.821 | 0.609 | 0.973 | 0.701 | 0.009 | 0.999 | 3.852 | 0.195 | 0.804 |
| S49 | 0.656 | 0.656 | 0.588 | 0.923 | 0.744 | 0.016 | 1.447 | 10.696 | 0.164 | 1.034 |
| S50 | 0.890 | 0.890 | 0.673 | 0.966 | 0.734 | 0.006 | 0.808 | 10.771 | 0.158 | 1.816 |
| S51 | 0.847 | 0.847 | 0.560 | 0.983 | 0.771 | 0.007 | 0.850 | 2.482 | 0.083 | 3.228 |
| S52 | 0.885 | 0.885 | 0.700 | 0.983 | 0.728 | 0.017 | 1.296 | 3.509 | 0.045 | 0.228 |
| S53 | 0.753 | 0.753 | 0.796 | 0.994 | 0.725 | 0.018 | 1.364 | 4.927 | 0.229 | 3.327 |
| S54 | 0.915 | 0.915 | 0.692 | 0.988 | 0.828 | 0.007 | 0.849 | 2.041 | 0.244 | 1.884 |
| S55 | 0.769 | 0.769 | 0.693 | 0.982 | 0.868 | 0.008 | 1.003 | 4.094 | 0.049 | 2.107 |
| S56 | 0.869 | 0.869 | 0.685 | 0.978 | 0.737 | 0.028 | 1.638 | 5.756 | 0.047 | 1.148 |
| S57 | 0.639 | 0.639 | 0.663 | 0.936 | 0.642 | 0.023 | 1.317 | 11.910 | 0.419 | 0.065 |
| S58 | 0.788 | 0.788 | 0.642 | 0.906 | 0.870 | 0.012 | 1.283 | 4.372 | 0.322 | 1.355 |
| S59 | 0.774 | 0.774 | 0.825 | 0.995 | 0.612 | 0.010 | 1.098 | 6.318 | 0.111 | 1.930 |
| S60 | 0.784 | 0.784 | 0.621 | 0.961 | 0.794 | 0.007 | 0.925 | 3.221 | 0.063 | 1.514 |
| S61 | 0.754 | 0.754 | 0.667 | 0.978 | 0.873 | 0.009 | 1.059 | 3.310 | 0.033 | 1.214 |
| S62 | 0.858 | 0.858 | 0.650 | 0.955 | 0.878 | 0.013 | 1.353 | 5.318 | 0.051 | 3.115 |
| S63 | 0.949 | 0.949 | 0.836 | 0.973 | 0.803 | 0.005 | 0.717 | 7.031 | 0.135 | 1.442 |
| S64 | 0.938 | 0.938 | 0.869 | 0.996 | 0.710 | 0.005 | 0.634 | 1.807 | 0.085 | 0.222 |
| S65 | 0.878 | 0.878 | 0.785 | 0.994 | 0.833 | 0.005 | 0.695 | 2.026 | 0.067 | 1.412 |
| S66 | 0.792 | 0.792 | 0.526 | 0.991 | 0.832 | 0.008 | 0.991 | 2.073 | 0.057 | 1.626 |
| S67 | 0.829 | 0.829 | 0.817 | 0.985 | 0.619 | 0.011 | 1.300 | 5.141 | 0.030 | 2.971 |
| S68 | 0.894 | 0.894 | 0.937 | 0.839 | 0.812 | 0.014 | 1.457 | 2.895 | 0.070 | 2.280 |
| S69 | 0.909 | 0.889 | 0.683 | 0.967 | 0.853 | 0.031 | 3.360 | 1.461 | 0.086 | 1.075 |
| S70 | 0.942 | 0.939 | 0.758 | 0.992 | 0.780 | 0.027 | 0.672 | 7.803 | 0.155 | 1.416 |
| mean | 0.824 | 0.824 | 0.710 | 0.938 | 0.748 | 0.013 | 1.295 | 4.178 | 0.154 | 1.872 |
| SD | 0.091 | 0.090 | 0.105 | 0.077 | 0.082 | 0.007 | 0.585 | 2.501 | 0.113 | 0.979 |

Comparing the results of the ground truth in the NW, the cardiac frequency index from the pupillary response indicates a strong correlation for all parameters where r=0.850±0.057 for VLF power; r=0.864±0.062 for LF power; r=0.855±0.066 for HF power; r=0.784±0.073 for the VLF/HF power ratio; and r=0.791±0.077 for the LF/HF power ratio. The difference between the mean error of all parameters was low with ME=0.457±0.313 for the VLF power; ME=0.506±0.292 for the LF power; ME=0.546±0.435 for the HF power; ME=0.692±0.436 for the VLF/HF power ratio, and ME=0.692±0.467 for the LF/HF power ratio.

### Real-Time System for Detecting the Cardiac Frequency Domain Parameters

The real-time system for detecting information of the cardiac frequency domain was developed based on capturing and processing of pupil images. This system may include an infrared webcam, near IR (Infra-Red light) illuminator (IR lamp) and personal computer for analysis.

The infrared webcam was divided into two types, the fixed type, which is a common USB webcam, and the portable type, which are represented by wearable devices. The webcam was a HD Pro C920 from Logitech Inc. converted into an infrared webcam to detect the pupil area.

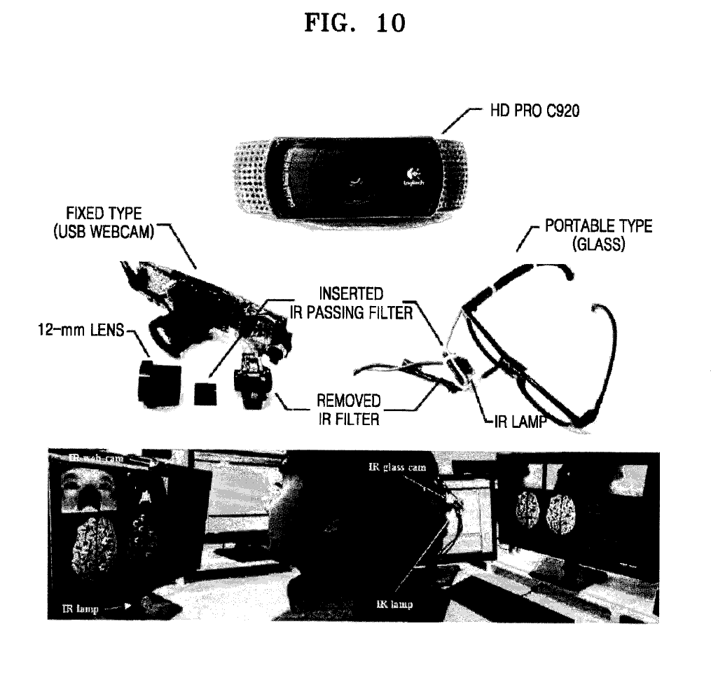

The IR filter inside the webcam was removed and an IR passing filter used for cutting visible light from Kodac Inc., was inserted into the webcam to allow passage of IR wavelength longer than 750 nm, as shown in FIG. 10. The 12-mm lens inside the webcam was replaced with a 3.6-mm lens to allow for focusing on the image when measuring the distance from 0.5 m to 1.5 m.

FIG. 10 shows an infrared webcam system for taking pupil images.

The conventional 12 mm lens of the USB webcam shown in FIG. 12 was replaced with a 3.6 mm lens so that the subject could be focused when a distance of 0.5 m to 1.5 m was photographed.

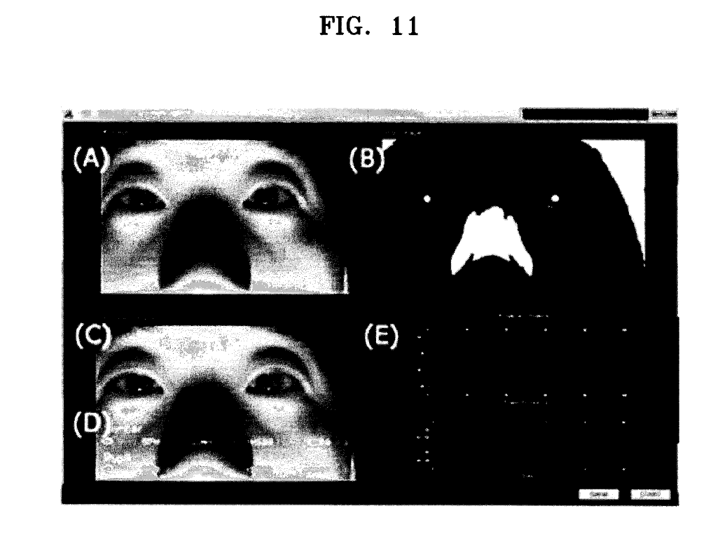

FIG. 11 shows an interface screen of a real-time system for detecting and analyzing a biological signal from an infrared webcam and a sensor, where (A): Infrared pupil image (input image), (B): binarized pupil image, (C): Detecting the pupil area, (D): Output of cardiac frequency parameters: (E): graphs of parameters of LF power, LF power, HF power, VLF/HF ratio, and LF/HF ratio).

As described in the above, the present invention develops and provides an advanced method for measurements of human vital signs from moving images of the pupil. Thereby, the measurement of parameters in cardiac frequency domain can be performed by using a low-cost infrared webcam system that monitored pupillary response.

This result was verified for both the conditions of noise (MNC and NMC) and various physiological states (variation of arousal and valence level by emotional stimuli of sound) for seventy subjects.

The research for this invention examined the variation in human physiological conditions caused by the stimuli of arousal, relaxation, positive, negative, and neutral moods during verification experiments. The method based on pupillary response according to the present invention is an advanced technique for vital sign monitoring that can measure vital signs in either static or dynamic situations.

The present invention may be applied to various industries such as U-health care, emotional information and communication technology (ICT), human factors, human computer interfaces (HCIs), and security that require vital signal monitoring (VSM) technology.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A method of detecting frequency-domain cardiac information, the method comprising:
acquiring moving images of a pupil from a subject;
extracting a pupil size variation (PSV) from the moving images;
extracting a heart rate variability (HRV) spectrum by performing a processing procedure including frequency-analysis of the PSV; and
calculating at least one power of at least one of a plurality of frequency bands from the HRV spectrum.

2. The method of claim 1, wherein the processing procedure includes filtering using a band pass filter (BPF) and frequency analysis using fast Fourier transformation (FFT).

3. The method of claim 2, wherein the plurality of frequency bands is the same as a plurality of frequency bands extracted from electrocardiogram (ECG) signals.

4. The method of claim 3, wherein the plurality of frequency bands include at least one of a very low frequency (VLF) in a range of 0.0033 Hz to 0.04 Hz, a low frequency (LF) in a range of 0.04 Hz to 0.15 Hz, and a high frequency (HF) in a range of 0.15 Hz to 0.4 Hz.

5. The method of claim 1, wherein the plurality of frequency bands include at least one of a very low frequency (VLF) in a range of 0.0033 Hz to 0.04 Hz, a low frequency (LF) in a range of 0.04 Hz to 0.15 Hz, and a high frequency (HF) in a range of 0.15 Hz to 0.4 Hz.

6. The method of claim 2, wherein the plurality of frequency bands include at least one of a very low frequency (VLF) in a range of 0.0033 Hz to 0.04 Hz, a low frequency (LF) in a range of 0.04 Hz to 0.15 Hz, and a high frequency (HF) in a range of 0.15 Hz to 0.4 Hz.

7. A system performing the method of claim 1, the system comprising:
a video capturing unit configured to capture the moving images of the subject; and
a computer architecture based analyzing system, including analysis tools, configured to process and analyze the moving images, and calculate at least one of the powers.

8. The system of claim 7, wherein the analyzing system performs filtering using a band pass filter (BPF) and frequency analysis using fast Fourier transformation (FFT).

9. The system of claim 8, wherein the plurality of frequency bands is the same as a plurality of frequency bands extracted from electrocardiogram (ECG) signals.

10. The system of claim 7, wherein the plurality of frequency bands include at least one of a very low frequency (VLF) in a range of 0.0033 Hz to 0.04 Hz, a low frequency (LF) in a range of 0.04 Hz to 0.15 Hz, and a high frequency (HF) in a range of 0.15 Hz to 0.4 Hz.

11. The system of claim 8, wherein the plurality of frequency bands include at least one of a very low frequency (VLF) in a range of 0.0033 Hz to 0.04 Hz, a low frequency (LF) in a range of 0.04 Hz to 0.15 Hz, and a high frequency (HF) in a range of 0.15 Hz to 0.4 Hz.

12. The system of claim 9, wherein the plurality of frequency bands include at least one of a very low frequency (VLF) in a range of 0.0033 Hz to 0.04 Hz, a low frequency (LF) in a range of 0.04 Hz to 0.15 Hz, and a high frequency (HF) in a range of 0.15 Hz to 0.4 Hz
